(19) United States
(12) Patent Application Publication
(10) Pub. No.: US 2019/0309014 A1
(43) Pub. Date: Oct. 10, 2019

(54) PROCESS FOR PREPARATION OF ICATIBANT ACETATE

(71) Applicant: Emcure Pharmaceuticals Limited, Bhosari, Pune (IN)

(72) Inventors: Mukund Keshav Gurjar, Bhosari, Pune (IN); Narendra Kumar Tripathy, Bhosari, Pune (IN); Chinmoy Mriganka Pramanik, Bhosari, Pune (IN); Ramesh Dhondikubeer, Bhosari, Pune (IN)

(73) Assignee: Emcure Pharmaceuticals Limited, Bhosari, Pune (IN)

(21) Appl. No.: 16/314,889

(22) PCT Filed: Jul. 3, 2017
(86) PCT No.: PCT/IB2017/054003

§ 371 Date: Jan. 3, 2019

(30) Foreign Application Priority Data
Jul. 4, 2016 (IN) 201621022862
Aug. 1, 2016 (IN) 201621026226

(51) Int. Cl. C07K 1/10; C07K 7/06; C07K 7/08; C07K 5/09; C07K 5/078; C07K 1/06

(52) U.S. Cl. CPC C07K 1/10; C07K 7/06; C07K 1/061; C07K 5/0817; C07K 5/06165; C07K 7/08

(57) ABSTRACT

The invention relates to an improved method for a 5+3+2 solution phase syntheses of Icatibant acetate (1) comprising coupling of suitably protected peptide fragments which on deprotection followed by treatment with acetic acid provide Icatibant acetate (1) having desired purity.

This application claims the benefit of Indian Provisional Applications No. IN201621022862 (filed on Jul. 4, 2016), and IN201621026226 (filed on Aug. 1, 2016), which are hereby incorporated by reference in entirety.

### FIELD OF THE INVENTION

The present invention relates to an improved process for solution phase synthesis of a decapeptide, Icatibant acetate comprising coupling of suitably protected polypeptide fragments by a 5+3+2 strategy, followed by deprotection and acetic acid treatment to afford the desired polypeptide, Icatibant acetate (1).

### BACKGROUND OF THE INVENTION

Icatibant acetate (1), chemically known as acetate salt of D-Arginyl-L-arginyl-L-prolyl-L[(4R)-(4-hydroxyprolyl)-glycyl-L[(3-(2-thienyl)alanyl)]-L-seryl-D-(1,2,3,4-tetrahydroisoquinolin-3-ylcarbonyl)-L[(3 aS,7aS)-octahydroindol-2-ylcarbonyl]-L-arginine, is a peptidomimetic decapeptide drug which is a selective and specific antagonist of bradykinin B2 receptors. It has been approved by the European Commission for the symptomatic treatment of acute attacks of hereditary angioedema (HAE) in adults with C1-esterase inhibitor deficiency.

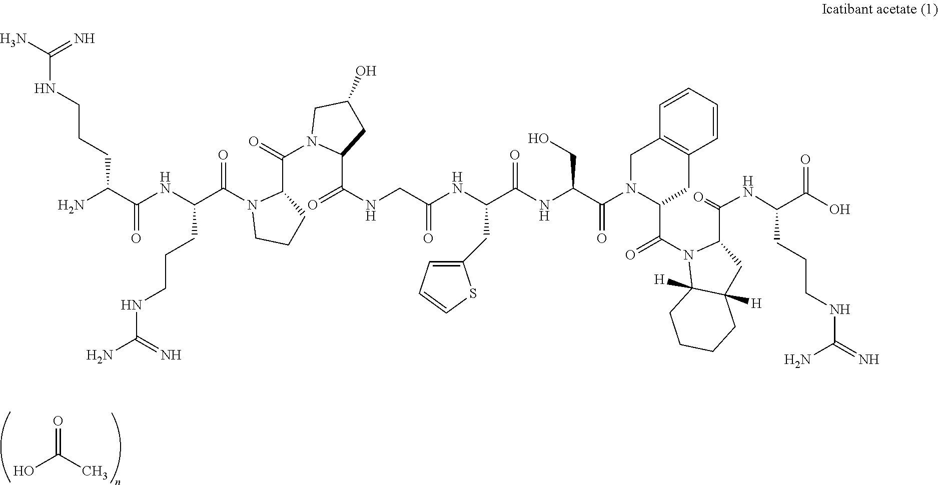

Icatibant acetate, developed by Shire Orphan Therapies Inc. with proprietary name Firazyr was first approved by USFDA on Aug. 25, 2011 as a subcutaneous injection with strength equivalent to 30 mg base/3 ml.

U.S. Pat. No. 5,648,333 discloses a process for preparation of the active ingredient comprising stepwise synthesis using a peptide synthesizer by Fmoc method on a p-benzyloxybenzyl alcohol resin esterified with Fmoc-Arg(Mtr)-OH. In each case, the amino acid derivative having a free carboxyl group for activation with HOBT was weighed into the cartridges of the synthesizer. The pre-activation of these amino acids was carried out directly in the cartridges by dissolving in DMF and adding diisopropylcarbodiimide in DMF. The HOBT esters of other amino acids were dissolved in NMP and then similarly coupled to the resin previously deblocked using piperidine in DMF, similar to the amino acids pre-activated in situ. After completion of the synthesis, the peptide was split off from the resin using thioanisole and ethanedithiol as cation entrainers, with simultaneous removal of the side chain protecting groups using trifluoroacetic acid. The residue obtained after stripping off the trifluoroacetic acid required repeated digestion with ethyl acetate for purification. The partly purified compound was further purified by chromatography using 10% acetic acid. The fractions containing the pure peptide were combined and freeze-dried.

CN102532267B discloses a similar method for solid phase synthesis of Icatibant which involves use of Fmoc-Arg(Pbf)-OH and a 2-chlorotrityl chloride resin for preparation of Fmoc-Arg(Pbf)-CTC resin and synthesis of Icatibant-CTC resin using the same by sequential coupling of the requisite amino acids. Further separation of the crude peptide from the resin and purification provided Icatibant.

CN103992383 discloses a process wherein a combination of solid and solution phase peptide synthesis methods is used to obtain Icatibant. The method specifically comprises synthesizing a fragment Boc-D-Arg-Arg-OH.2HCl by a liquid phase, followed by sequential coupling of relevant Fmoc protected amino acids by solid-phase synthesis method, wherein coupling of the last two amino acids is performed by the fragment Boc-D-Arg-Arg-OH.2HCl. Further cleavage of the peptide from the resin, purification, desalination and lyophilization yielded Icatibant. WO2015128687 discloses a continuous flow method for the solid phase synthesis of various polypeptides including Icatibant.

It would be evident from a review of prior art that most of the synthetic methods disclosed in the aforementioned references involve solid phase syntheses or a combination of solid and solution phase peptide syntheses wherein a dipeptide is synthesized by solution phase method and the other octapeptide fragment is constructed through solid phase synthesis.

However, these methods utilize expensive resins, costly reagents, elaborate deprotection and separation procedures at various intermediate stages of synthesis. Further, these methods involve use of Fmoc/tert-butyl protected amino acids in three to four fold excess, necessitating complex purification procedures to separate the product from the impurities. These additional steps before isolation render these processes extremely exorbitant for large scale industrial production of the desired product.

Solution phase synthesis methods for peptides, on the other hand, comprise independent synthesis of amino acids segments or blocks, followed by condensation of various segments in the desired sequence in solution. Such processes are comparatively economical and hence more suited for synthesis on industrial scale. Hence, there is a need for a convenient and economical synthetic process for Icatibant acetate which involves solution phase synthetic approach comprising practical synthesis of suitable fragments utilizing specific, easily removable protecting groups followed by their condensation, deprotection reactions with the use of mild and selective reagents to achieve the desired conversions.

The present inventors have developed an economical and convenient process for solution phase synthesis of Icatibant acetate (1) which provides the desired molecule in good yield overcoming the problems faced in the prior art. The use of 5+3+2 strategy comprising synthesis of small peptide fragments, in combination with highly specific protection and deprotection methods and a facile condensation of the fragments facilitates in obtaining the desired molecule in fewer synthetic steps with significant yield improvement as compared to prior art processes.

### OBJECT OF THE INVENTION

An objective of the present invention is to provide an industrially applicable, convenient process for synthesis of Icatibant acetate (1), which avoids use of expensive resins and costly reagents that are used in solid phase peptide synthesis methods.

Another object of the invention relates to a 5+3+2 solution phase synthesis of Icatibant acetate comprising easily detachable, labile protecting groups and mild reaction conditions for coupling the fragments to provide the final compound possessing desired purity.

### SUMMARY OF THE INVENTION

An aspect of the invention relates to a 5+3+2 solution phase synthetic process for Icatibant acetate (1) comprising reaction of H-Thia-Ser(O-tBu)-D-Tic-Oic-Arg(Pbf)-OtBu (fragment A) with Fmoc-Hyp-Gly-OH (fragment B) in presence of a coupling agent, in an organic solvent and a base to give the heptapeptide intermediate H-Hyp(OP)-Gly-Thia-Ser(OP)-D-Tic-Oic-Arg(Pbf)-O-tBu (21), further coupling with Boc-D-Arg(Pbf)-Arg(Pbf)-Pro-OH (fragment C) in presence of a coupling agent, in an organic solvent and a base to provide the decapeptide Boc-D-Arg(Pbf)-Arg(Pbf)-Pro-Hyp-Gly-Thia-Ser(O-tBu)-D-Tic-Oic-Arg(Pbf)-O-tBu (29), subsequent deprotection and treatment with acetic acid to provide lcatibant acetate (1) having desired purity.

The objectives of the present invention will become more apparent from the following detailed description.

### DETAILED DESCRIPTION OF THE INVENTION

The present inventors, in their quest for developing a convenient, industrially viable process by solution phase synthetic strategy for Icatibant acetate, surprisingly found that synthesis of suitably protected polypeptide fragments, followed by facile condensation reactions and deprotection provided the desired polypeptide in good yield with significant control over formation of impurities.

The inventors also unexpectedly found that most of the intermediates in the said strategy were obtained as solids, due to which various laborious and cumbersome intermediate isolation and purification steps were avoided. The reduction in the number of unit steps not only improved yield significantly for the desired compound but also led to a convenient and economical synthetic process for Icatibant acetate which could easily be scaled up for commercial production.

Further, during the synthesis of pentapeptide and dipeptide fragments, respective allyl (—CH₂—CH═CH₂) protection of the indolyl and glycyl carbonyl groups which could be deprotected using Palladium (0) catalyst avoided use of bases like lithium hydroxide, thus significantly minimizing the problems of racemization which are very commonly observed in the solution phase synthesis of polypeptides. The instant strategy also comprises selective and specific, yet labile protecting groups at different stages, which are deprotected using mild acids, that do not adversely affect the chirality of the amino acids and intermediates in the synthetic sequence.

Outline of the 5+3+2 synthetic strategy for lcatibant is provided in Scheme-1. Synthesis of the respective fragments is disclosed in the synthetic schemes as given below.
a) Pentapeptide fragment A: Scheme-2;
b) Dipeptide fragment B and Heptapeptide intermediate: Scheme-3;
c) Tripeptide fragment C: Scheme-4 and
d) Coupling of the heptapeptide with fragment C, deprotection and acetic acid treatment to give Icatibant acetate: Scheme-5.

### Abbreviations

### Fmoc=Flourenylmethoxycarbonyl

### Tbu=Tert-butyl

Pbf=2,2,4,6,7-Pentamethyldihydrobenzofuran-5-sulfonyl

### THF=Tetrahydrofuran

### DMF=N, N-Dimethylformamide

DMSO=Dimethyl sulfoxide

### DMAc=N, N-Dimethylacetamide

### NMM=N-methylmorpholine

### TEA=Triethylamine

### DEA=Diethylamine

### Bn=Benzyl

TFA=Trifluoroacetic acid

### EDT=Ethanedithiol

### TIS=Triisopropylsilane

### HOBt=1-Hydroxybenzotriazole

### DCM=Dichloromethane

EDAC=1-Ethyl-3-(3-dimethylaminopropyl)carbodiimide
HPLC=High performance liquid chromatography
TLC=Thin layer chromatography
PTSA=p-toluene sulfonic acid
MTBE=Methyl tertiary butyl ether
HCl=Hydrochloric acid

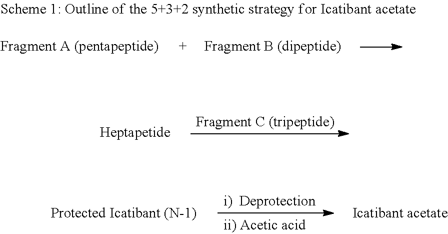

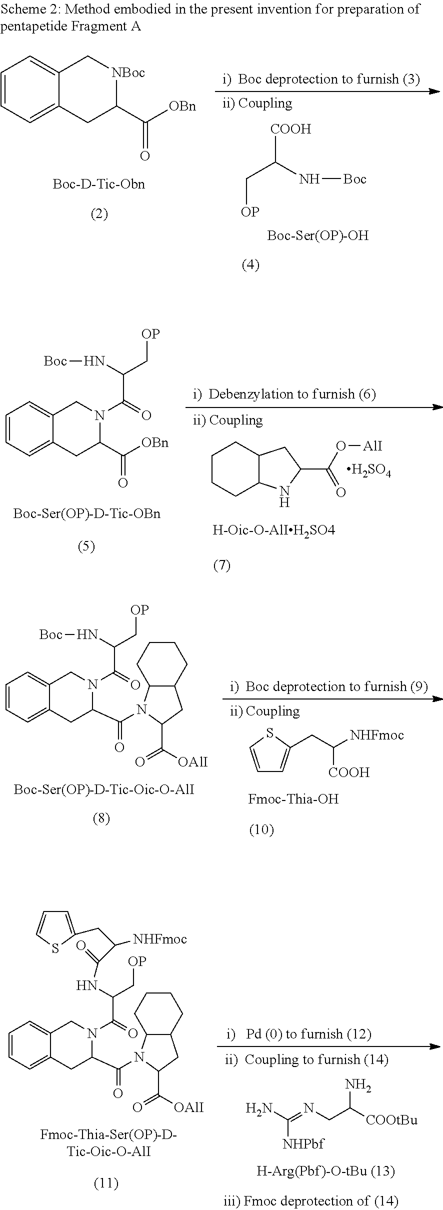

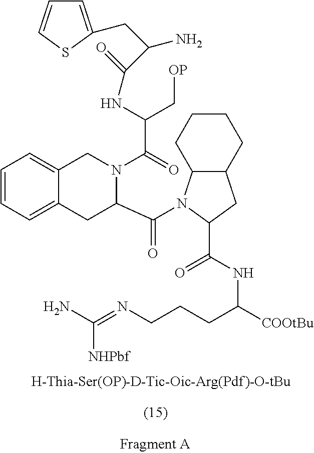

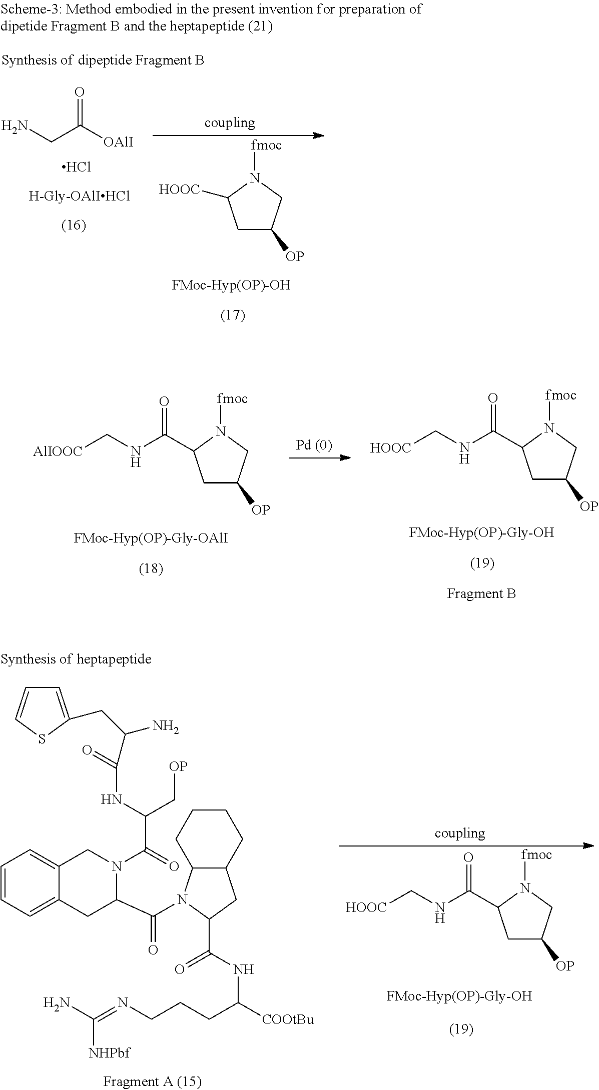

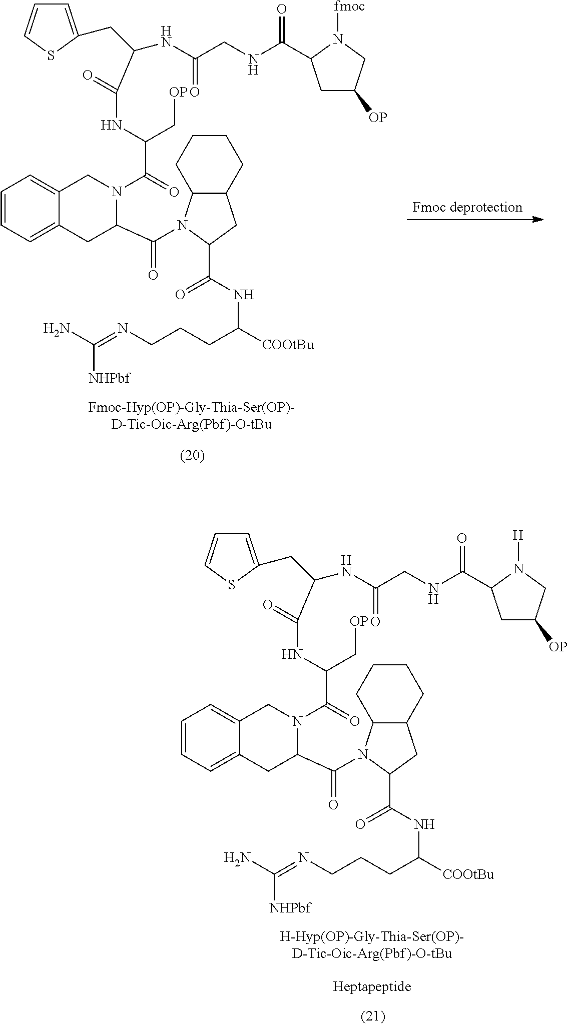

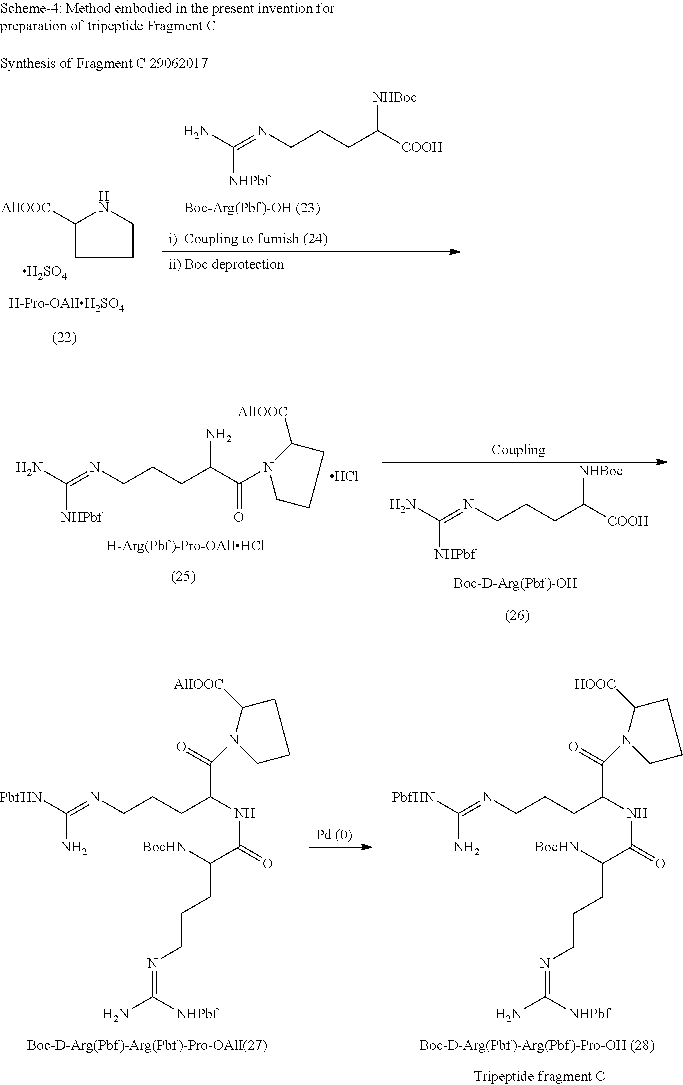

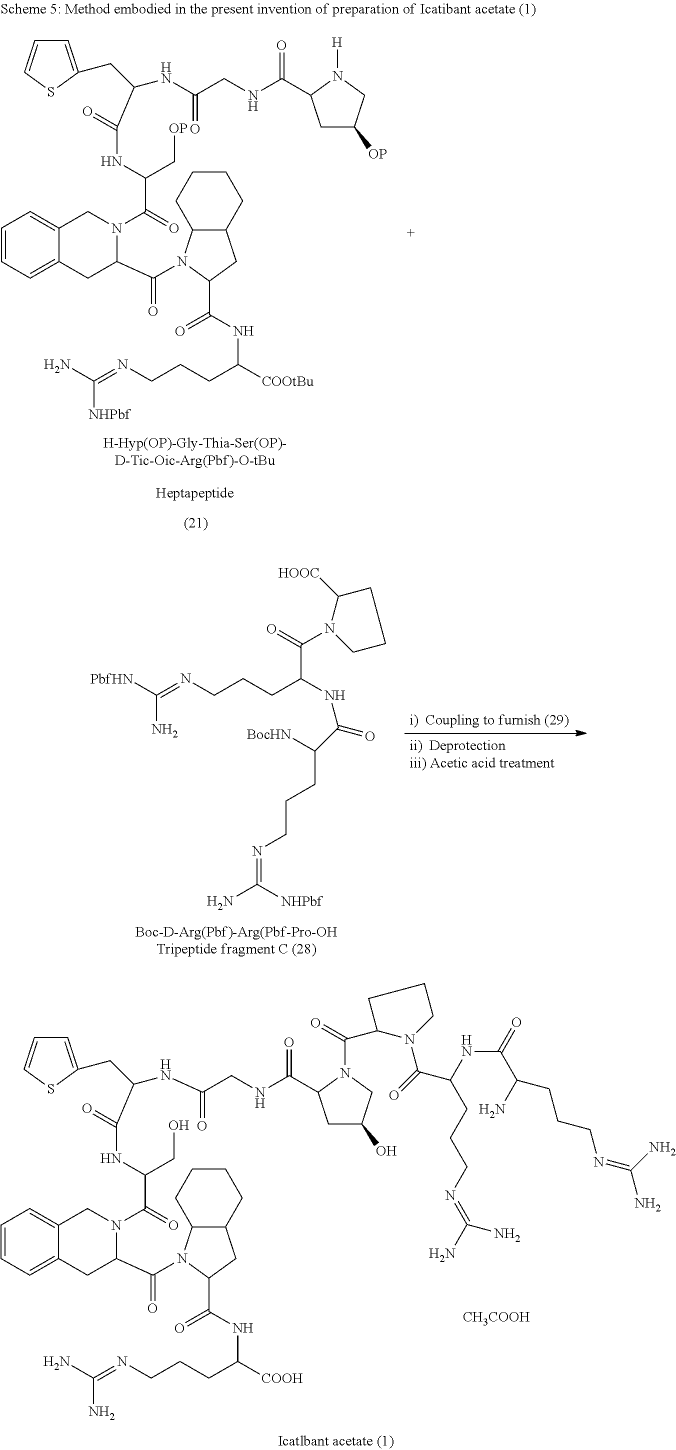

In an embodiment, the benzyl ester, Boc-D-Tic-OBn (2) was subjected to Boc deprotection at ambient temperature using suitable acid and a solvent to give H-D-Tic-OBn (3) as acid-salt, which was then treated with a carbonate or bicarbonate base to give the free base (3), prior to further reaction. Compound (3), when coupled with Boc-Ser(OP)-OH (4) in presence of a coupling agent and a suitable organic solvent in the temperature range of 0-30° C., gave Boc-Ser(OP)-D-Tic-OBn (5). After completion of the reaction, as monitored by HPLC, the reaction mixture was filtered, filtrate was concentrated and water was added to the residue, followed by addition of hydrocarbon solvent such as hexane, heptane, toluene etc. or mixtures thereof. Filtration, layer separation and concentration of the organic layer provided (5).

Optionally the acid-salt of H-D-Tic-OBn (3) was coupled with Boc-Ser(OP)-OH (4) in presence of a coupling agent, a base like NMM and a suitable organic solvent such as DMF, in the temperature range of 0-30° C. After completion of the reaction, as monitored by HPLC, the reaction mixture was quenched with 0.5 N hydrochloric acid. Extraction with ethyl acetate, followed by separation and concentration of the organic layer gave the desired compound (5).

The group P herein is a protecting group selected from the group comprising H, tert-butyl, tert-butyldimethyl silane, triethyl silane, methoxymetrhyl, methoxy ethoxymethyl etc.

Benzyl deprotection of (5) using metal catalysts such as Pd/C and a suitable solvent under hydrogenation conditions with hydrogen pressure in the range of 3-10 Kg/cm², at ambient temperature, afforded Boc-Ser(OP)-D-Tic-OH (6). After completion of benzyl deprotection as monitored by HPLC, the reaction mass was filtered and concentrated to give (6). Coupling of (6) with H-Oic-OAll (7) in presence of a coupling agent using an organic solvent in the temperature range of 0-30° C. gave Boc-Ser(OP)-D-Tic-Oic-OAll (8). After completion of the reaction, as monitored by HPLC, the reaction mixture was concentrated and water was added to the residue, followed by addition of hydrocarbon solvent such as hexane, heptane, toluene etc. or mixtures thereof. Filtration, layer separation and concentration of the organic layer provided (8).

Optionally the acid-salt of (7), H-Oic-OAll.H₂SO₄ was coupled with Boc-Ser(OP)-D-Tic-OH (6) in presence of a coupling agent, a base like NMM and a suitable organic solvent such as DMF. After completion of the reaction, as monitored by HPLC, the reaction mixture was quenched with 0.5 N hydrochloric acid and filtered. The solid obtained was dissolved in dichloromethane and the resulting mixture was washed with 0.5 N hydrochloric acid and 5% sodium bicarbonate solution. Separation and concentration of the organic layer gave the desired compound (8).

Boc deprotection of (8) using a suitable acid such as trifluoroacetic acid and an organic solvent at ambient temperature afforded H-Ser(OP)-D-Tic-Oic-OAll (9). After complete deprotection of the Boc group, as monitored by HPLC, reaction mass was quenched with water and neutralized. Extraction with dichloromethane, separation and concentration of the organic layer gave (9).

Optionally, Boc deprotection of (8) was carried out using mineral acid like HCl in an organic solvent such as acetonitrile. After complete deprotection of the Boc group, as monitored by HPLC, reaction mass was concentrated and treated with hydrocarbon solvents such as n-hexane, heptanes to give (9).

Coupling of (9) with Fmoc-Thia-OH (10) in presence of a coupling agent in a suitable organic solvent like acetonitrile furnished Fmoc-Thia-Ser (OP)-D-Tic-Oic-OAR (11). After completing the reaction, as monitored by HPLC, the reaction mass was concentrated and organic solvent such as ethyl acetate was added to the residue, followed by addition of bicarbonate solution. Separation and concentration of the organic layer gave (11).

Optionally, the coupling of compounds (9) and (10) was carried out in presence of base like NMM using solvent such as DMF. After completion, as monitored by HPLC, the reaction mixture was quenched with hydrochloric acid solution, and filtered. The solid thus obtained was washed with dilute acid, base and dried to give (11) which was optionally purified using column chromatographic techniques.

Allyl deprotection of (11) using triphenylphosphine palladium (0) catalyst in presence of morpholine or sodium 2 ethyl hexanoate, at ambient temperature provided Fmoc-Thia-Ser(OP)-D-Tic-Oic-OH (12). After completion of allyl deprotection, as monitored by HPLC, the reaction mass was concentrated and residue was dissolved in organic solvent. Neutralization of the mixture, followed by extraction with organic solvent selected from ethers. Separation of the organic layer, acidification of the aqueous layer and filtration gave a solid. Dissolving the solid so obtained in organic solvent selected from esters, removal of moisture and concentration of the organic layer gave (12).

Coupling of (12) with H-Arg(Pbf)-O-tBu (13), in presence of a coupling agent and a base in a suitable organic solvent furnished Fmoc-Thia-Ser(OP)-D-Tic-Oic-Arg(Pbf)-O-tBu (14). After completion of the reaction, as monitored by HPLC, the reaction mass was quenched with acid, and filtered to give (14).

Fmoc deprotection of (14) using a suitable base and organic solvent afforded H-Thia-Ser (OP)-D-Tic-Oic-Arg(Pbf)-OtBu(15), labeled as Fragment A. After complete deprotection of the Fmoc group, as monitored by HPLC, the reaction mixture was quenched with acid and the resulting mass was extracted with organic solvents selected from ethers. Separation of the organic layer, extracting the aqueous layer with another organic solvent selected from esters such as ethyl acetate, concentration of the separated organic layer and treatment of the residue with hydrocarbon solvent provided fragment A.

In another embodiment, the allyl ester of Glycine HCl, H-Gly-OAll.HCl (16) was coupled with Fmoc-Hyp(OP)-OH (17) in a suitable solvent in presence of a coupling agent and a base in the temperature range of 0-30° C. to give Fmoc-Hyp(OP)-Gly-OAll (18). After completion of the reaction, as monitored by HPLC, the reaction mass was quenched with acid, followed by filtration. Solid so obtained was optionally treated with hydrocarbon solvent like cyclohexane to give (18).

Allyl deprotection of (18) using Palladium (0) catalyst in presence of morpholine or sodium 2-ethylhexanoate in an organic solvent like MDC, THF provided the dipeptide Fmoc-Hyp(OP)-Gly-OH (19), labeled as Fragment B. After complete deprotection, as monitored by HPLC, the reaction mass was concentrated and residue was dissolved in water miscible organic solvent such as DMF. Neutralization, extraction with organic ether solvent, separation of the aqueous layer, followed by acidification, filtration gave (19).

The group P herein has the same meaning as defined earlier.

In yet another embodiment, H-Thia-Ser(OP)-D-Tic-Oic-Arg(Pbf)-OtBu (15), (Fragment A) was coupled with Fmoc-Hyp(OP)-Gly-OH (19) in presence of a coupling agent, a base and a suitable organic solvent in the temperature range of 0-30° C. to give Fmoc-Hyp(OP)-Gly-Thia-Ser (OP)-D-Tic-Oic-Arg(Pbf)-OtBu (20). After completion of the reaction, as monitored by HPLC, the reaction mass was quenched with acid followed by filtration. Organic solvent selected from halogenated hydrocarbons was added to the obtained solid, along with mild alkali solution. Separation and concentration of the organic layer gave (20).

Fmoc deprotection of (20) using a suitable base and organic solvent at ambient temperature afforded the heptapeptide fragment H-Hyp(OP)-Gly-Thia-Ser(OP)-D-Tic-Oic-Arg(Pbf)-OtBu (21). After completion of the reaction, as monitored by HPLC, the reaction mass was quenched with acid, and the acidified mixture was extracted with organic solvents selected from ethers. Separation of the organic layer, extracting the aqueous layer with another organic solvent selected from esters such as ethyl acetate gave an organic layer containing the desired compound. Concentration of the organic layer and optional treatment with hydrocarbon solvent such as toluene provided (21).

In a further embodiment, H-Pro-OAll (22) as free base or in the form of acid salt such as H-Pro-OAll.H₂SO₄ was coupled with Boc-Arg(Pbf)-OH (23) in presence of a coupling agent, a base and a suitable organic solvent in the temperature range of 0-30° C. to give Boc-Arg(Pbf)-Pro-OAll (24). After completion of the reaction, as monitored by HPLC, the reaction mass was quenched with acid, stirred and filtered to give (24) as a solid.

Boc deprotection of (24) using a suitable acid and an organic solvent at 25 to 30° C. afforded H-Arg(Pbf)-Pro-OAll (25) as acid salt. After complete deprotection, filtration and concentration of the reaction mixture provided the desired compound (25).

Coupling of (25) with Boc-D-Arg(Pbf)-OH (26) in presence of a coupling agent and a base in a suitable organic solvent in the temperature range of 0-30° C. gave Boc-D-Arg(Pbf)-Arg(Pbf)-Pro-OAll (27). After completion of the reaction, as monitored by HPLC, the reaction mass was quenched with acid, stirred and filtered to give (27) as solid.

Allyl deprotection of (27) using Palladium (0) catalyst in presence of morpholine or sodium 2-ethylhexanoate in an organic solvent like MDC, THF provided the tripeptide, Boc-D-Arg(Pbf)-Arg(Pbf)-Pro-OH (28), Fragment C. After complete deprotection, as monitored by HPLC, the reaction mass was concentrated and residue was dissolved in water miscible organic solvent. Neutralization, extraction with organic ether solvent, separation of the aqueous layer, followed by acidification, filtration gave (28) as solid.

In yet another embodiment, coupling of heptapeptide fragment (21) and Fragment C (28) in presence of a coupling agent, a base, and a suitable organic solvent in the temperature range of 0-30° C. furnished the decapeptide (29). After completion of the reaction, as monitored by HPLC, the reaction mass was quenched with acid, stirred and filtered to give (29) as solid, which was optionally purified using chromatographic techniques.

Compound (29) was subjected to deprotection reaction using TFA, TES etc. at ambient temperature. After completion of the reaction, as monitored by HPLC, concentration of the reaction mixture and treatment of resulting oily residue with organic solvent selected from a group of ethers such as diethyl ether, methyl tertiary butyl ether etc. provided a solid. Purification of the solid using chromatographic techniques, followed by acetic acid treatment of the desired fractions afforded Icatibant acetate (1).

Organic solvents that can be used are selected from the group comprising aprotic solvents such as nitriles chlorinated solvents, ethers, and esters. Examples of these solvents are methylene chloride, chloroform, dichloroethane, dimethylforinamide, dimethylacetamide, tetrahydrofuran, ethyl acetate, 1-methyl-2-pyrrolidinone, acetonitrile, or combinations thereof.

Coupling agents are selected from the group comprising substituted carbodiimides such as diisopropylcarbodiimide, dicyclohexylcarbodiimide, BOP (Benzotriazol-1-yloxy-tris(dimethylamino)-phosphonium hexafluorophosphate), PyBOP (Benzotriazol-1-yloxy-tripyrrolidino-phosphoniumhexafluorophosphate), PyBrOP (Bromotripyrrolidino phosphonium hexafluorophosphate), PyAOP (7-Aza-benzotriazol-1-yloxy-tripyrrolidinophosphonium hexafluorophosphate), DEPBT (3-(Diethoxyphosphoryloxy)-1,2,3-benzo[d]triazin-4(3H)-one), TBTU (2-(1H-Benzotriazol-1-yl)-N,N,N′,N′-tetramethylaminium tetrafluoroborate), HBTU (2-(1H-Benzotriazol-1-yl)-N,N,N′,N′-tetramethylaminium hexafluoroborate), HATU (2-(7-Aza-1H-benzotriazol-1-yl)-N,N,N′,N′-tetramethylaminium hexafluorophosphate), COMU (1-[1-(Cyano-2-ethoxy-2-oxoethylideneaminooxy)-dimethylamino-morpholino]-uroniumhexafluorophosphate), HCTU (2-(6-Chloro-1H-benzotriazol-1-yl)-N,N,N′,N′-tetramethylaminium hexafluorophosphate) and TFFH (Tetramethylfluoroformamidinium hexafluorophosphate).

The bases are selected from the group comprising of Diisopropyl ethyl amine (DIPEA), N-methylmorpholine (NMM), triethyl amine, Diethyl amine, N-methylmorpholine, piperidine, N-methylpyrrolidine.

The protecting group, denoted as P in the embodiments is selected from the group of H, tert-butyl, tert-butyldimethyl silane, triethyl silane, methoxymetrhyl, and methoxy ethoxymethyl.

The acid employed for deprotection is selected from the group comprising of trifluoroacetic acid, hydrochloric acid gas dissolved in ethyl acetate or dioxane.

### EXAMPLES

### Example 1: Synthesis of Boc-Ser(O-tBu)D-Tic-OBn (5)

HCl in acetonitrile (508 nil) was added to the stirred solution of Boc-D-Tic-OBn (2) (127.0 g) in acetonitrile (381 ml) and the mixture was stirred at 25-30° C. After complete deprotection of the Boc group, as monitored by HPLC, the reaction mass was filtered to give H-D-Tic-OBn.HCl.

Yield: 99.0 g (94.27%), Purity: 96% (HPLC)

Aqueous solution of sodium bicarbonate was added to H-D-Tic-OBn.HCl (50 g), mixture was stirred and extracted with ethyl acetate. Separation and concentration of the organic layer provided H-D-Tic-OBn (3, 43.5 g).

HOBt (41.55 g) EDAC.HCl (52.01 g) were added to the stirred solution of Boc-Ser(O-tBu)-OH (4) (47.28 g) in acetonitrile (150 ml) at 0° C., followed by addition of H-D-Tic-OBn (3, 43.5 g) in acetonitrile (100 ml). The reaction mass was stirred at 20 to 30° C., till completion of the reaction, as monitored by HPLC.

After completion, the reaction mixture was cooled, stirred, filtered, concentrated and water was added to the residue. Toluene (250 ml) was added to the resulting mixture, which was stirred at 20 to 30° C. The solid was filtered off and layers in the filtrate were separated. The organic layer was washed with 5% aqueous potassium hydrogen sulfate, and 5% aqueous sodium bicarbonate solution. If in case any emulsion was observed, it was filtered off. The organic layer, thus obtained was concentrated to give Boc-Ser(O-tBu)D-Tic-OBn (5).

Yield: 66.5 g, (79.12%), Purity: 92% (HPLC)

### Example 2: Preparation of Boc-Ser-(O-tBu)-D-Tic-Oic-OAll (8)

Palladium on carbon (10%, 50% moisture, 6.5 g) in water (6.5 ml) was added to the stirred solution of Boc-Ser-(O-tBu)-D-Tic-OBn (5, 65.0 g) in ethyl acetate (260 ml) and the reaction was continued under hydrogen pressure 5-6 Kg/cm² at ambient temperature. After complete deprotection of the benzyl group as monitored by HPLC, the reaction mass was filtered and concentrated to give Boc-Ser-(O-tBu)-D-Tic-OH (6) as solid.

Yield: 50.4 g, (94.17%), Purity: 90% (HPLC)

Compound (6, 50.0 g) was dissolved in acetonitrile (150 ml) and HOBt (27.3 g) was added to the reaction mixture, which was cooled to 0° C., followed by addition of EDAC.HCl (34.2 g). The reaction mixture was stirred at 0 to 5° C. and a solution of H-Oic-OAll (7, 22.2 g) in acetonitrile (150 ml) was added to it with continued stirring at the same temperature. After completion of the reaction, as monitored by HPLC, the reaction mass was concentrated and water was added to the residue. Toluene (250 ml) was added to the resulting mixture, which was stirred at 20 to 30° C. The solid was filtered off and layers in the filtrate were separated. The organic layer was washed with 5% aqueous sodium hydrogen sulfate, and 5% aqueous sodium bicarbonate solution. If in case any emulsion was observed, it was filtered off. The organic layer, thus obtained was concentrated to give Boc-Ser-(O-tBu)-D-Tic-Oic-OAll (8).

Yield: 30.0 g, (41.24%), Purity: 90.0% (HPLC)

### Example 3: Preparation of Fmoc-Thia-Ser(O-tBu)-D-Tic-Oic-OAll (11)

Trifluoroacetic acid (40 ml) was added to the stirred solution of Boc-Ser-(O-tBu)-D-Tic-Oic-OAll (8, 25 g) in dichloromethane (60 ml) and the reaction mixture was stirred at 0 to 10° C. After complete deprotection of the Boc group, as monitored by HPLC, reaction mass was quenched with water and neutralized using aqueous sodium bicarbonate. Extraction with dichloromethane, separation and concentration of the organic layer gave H-Ser-(O-tBu)-D-Tic-Oic-OAll (9, 19.5 g). HOBt (8.23 g) was added to the mixture of Fmoc-Thia-OH (10, 12.66 g) in acetonitrile (63 ml). The reaction mixture was cooled to 0° C. and EDAC.HCl (10.76 g) was further added to it. The resultant mixture was stirred at 0 to 5° C. and a solution of H-Ser-(O-tBu)-D-Tic-Oic-OAll (9, 19.0 g) in acetonitrile (190 ml) was added to it. The reaction was continued at 0 to 10° C. After completing the reaction, as monitored by HPLC, the reaction mass was concentrated and ethyl acetate was added to the residue, followed by addition of 5% aqueous sodium bicarbonate solution. Separation and concentration of the organic layer gave Fmoc-Thia-Ser(O-tBu)-D-Tic-Oic-OAll (11).

Yield: 31.66 g, (87.33%), Purity: 85% (HPLC)

### Example 4: Preparation of H-Thia-Ser(O-tBu)-D-Tic-Oic-Arg(Pbf)-OtBu (15), Fragment A

The solution of Fmoc-Thia-Ser(O-tBu)-D-Tic-Oic-OAll (11, 10.0 g) in dichloromethane (50 ml) was stirred and tetrakis(triphenylphosphine) Palladium (0) catalyst, (0.70 g) and sodium 2-ethylhexanoate (2.0 g) were added to it. Reaction mixture was stirred at 25 to 30° C. After complete deprotection of the allyl group, as monitored by HPLC, the reaction mass was concentrated and residue was dissolved in DMF (50 ml). Water, 5% aqueous sodium bicarbonate solution were added to the mixture followed by extraction with MTBE. The organic layer was separated and water and 0.5 N Hydrochloric acid were added to the aqueous layer till it was acidic, followed by stirring and filtration. The wet cake was dissolved in ethyl acetate. The aqueous layer, if any, was separated and the organic layer was concentrated to give Fmoc-Thia-Ser(O-tBu)-D-Tic-Oic-OH (12).

Yield: 8.0 g, (83.85%), Purity: 85.0% (HPLC)

Compound 12 (7.0 g) was dissolved in DMF (21 ml) and HOBT (1.89 g) was added to it. Reaction mixture was cooled to 0° C., and EDAC.HCl (2.38 g) was added to it. The resultant mixture was stirred at 0 to 5° C. and N-methylmorpholine (2.1 g) was added to it. H-Arg(Pbf)-OtBu.HCl (13, 4.34 g), along with DMF (7 ml) was then added to the stirred reaction mixture at 0 to 5° C. and the reaction was continued at 20 to 30° C. After completion of the reaction, as monitored by HPLC, the reaction mass was quenched with 0.5 N Hydrochloric acid stirred and filtered. The solid so obtained was washed with water, sodium bicarbonate solution and dried to give Fmoc-Thia-Ser(O-tBu)-D-Tic-Oic-Arg(Pbf)-O-tBu (14).

Yield: 10.5 g, (96.95%), Purity: 86.0% (HPLC)

Compound (14, 8.0 g) in DMF (40 ml) was treated with triethylamine (6.18 g) at 20 to 30° C. After complete deprotection of the Fmoc group, as monitored by HPLC, the reaction mixture was quenched with 0.5 N hydrochloric acid till it was acidic and the resulting mass was extracted with methyl tertiary butyl ether. The organic layer was separated. Water was added to the aqueous layer followed by extraction with ethyl acetate. Separation and concentration of the organic layer gave a residue, which when treated with toluene provided H-Thia-Ser(O-tBu)-D-Tic-Oic-Arg(Pbf)-OtBu (15), Fragment A.

Yield: 6.0 g, (87.33%), Purity: 82% (HPLC)

### Example 5: Preparation of Fmoc-Hyp-Gly-OH (19), Fragment B

HOBt (4.77 g) was added to the stirred solution of Fmoc-Hyp-OH (17, 10.0 g) in DMF (30 ml). Reaction mixture was cooled to 0° C., and EDAC.HCl (7.05 g) and H-Gly-OAll.HCl (16, 5.6 g) in DMF (25 ml) were added to it, followed by addition of N-methylmorpholine (3.70 g). The reaction mixture was stirred at 20 to 30° C. After completion of the reaction, as monitored by HPLC, the reaction mass was quenched with 0.5 N Hydrochloric acid, followed by stirring and filtration. The solid thus obtained was washed with water followed by treatment with cyclohexane to give Fmoc-Hyp-Gly-OAll (18).

Yield: 11.1 g, (87.12%), Purity: 92% (HPLC)

The stirred solution of compound (18, 10.0 g) in MDC, (50 ml) was treated with tetrakis(triphenylphosphine) Palladium (0) (1.28 g) and sodium 2-ethylhexanoate (4.64 g) in tetrahydrofuran (175 ml) at 20 to 30° C. After completion of the reaction, as monitored by HPLC, the reaction mass was concentrated and residue was dissolved in DMF (50 ml), followed by addition of 5% Sodium bicarbonate solution and water. The resulting mass was extracted with methyl tertiary butyl ether. The organic layer was separated. Water was added to the aqueous layer followed by addition of 0.5 N hydrochloric acid till it was acidic. Stirring and filtration gave a solid which was washed with water and dried to give Fmoc-Hyp-Gly-OH (19), Fragment B.

Yield: 7.1 g, (77.85%), Purity: 88% (HPLC)

### Example 6: Preparation of Heptapeptide Intermediate, H-Hyp-Gly-Thia-Ser(O-tBu)-D-Tic-Oic-Arg(Pbf)-O-tBu (21)

HOBt (1.05 g) was added to the stirred solution of Fmoc-Hyp-Gly-OH (19, 2.26 g) in DMF (20 ml) The reaction mixture was cooled to 0° C., and EDAC.HCl (1.32 g) and N-methylmorpholine (1.16 g) were added to it. H-Thia-Ser(O-tBu)-D-Tic-Oic-Arg(Pbf)-O-tBu, Fragment A (15, 5.0 g), and DMF (15 ml), were added to the mixture stirred at 0 to 5° C. and the reaction was continued at 20 to 30° C. After completion of the reaction, as monitored by HPLC, the reaction mass was quenched with 0.5 N hydrochloric acid followed by stirring and filtration. The solid so obtained was washed with water, and sodium bicarbonate solution and dichloromethane were added to it. The organic layer was separated and concentrated to give Fmoc-Hyp-Gly-Thia-Ser(O-tBu)-D-Tic-Oic-Arg(Pbf)-O-tBu (20).

Yield: 5.6 g, (85.23%), Purity: 89% (HPLC)

Compound (20, 5.0 g) in DMF (25 ml) was treated with triethylamine (3.4 g) at 20 to 30° C. After complete deprotection of the Fmoc group, as monitored by HPLC, the reaction mixture was quenched with 0.5 N hydrochloric acid till it was acidic and the resulting mass was extracted with methyl tertiary butyl ether. The organic layer was separated. Water was added to the aqueous layer followed by extraction with ethyl acetate. Separation and concentration of the organic layer gave a residue, which when treated with toluene provided H-Hyp-Gly-Thia-Ser(O-tBu)-D-Tic-Oic-Arg(Pbf)-O-tBu (21).

Yield: 3.19 g, (72.86%), Purity: 76% (HPLC)

### Example 7: Preparation of Boc-D-Arg(Pbf)-Arg(Pbf)-Pro-OH (28), Fragment C

HOBt (18.9 g) was added to the stirred solution of Boc-Arg(Pbf)-OH (23, 50.0 g) in DMF (200 ml). The reaction mixture was cooled to 0° C., and EDAC.HCl (36.4 g) and N-methylmorpholine (19.2 g) were added to it. H-Pro-OAll.H₂SO₄ (22, 48.1 g) in DMF (50 ml) was added to the mixture stirred at 0 to 5° C. and the reaction was continued at 20 to 30° C. After completion of the reaction, as monitored by HPLC, the reaction mass was quenched with 0.5 N hydrochloric acid followed by stirring and filtration. The solid so obtained was washed with water, 7% sodium bicarbonate solution and dried to give Boc-Arg(Pbf)-Pro-OAll (24).

Yield: 59.2 g, (93.93%)

Acetonitrile in HCl (165 ml) was added to the stirred solution of compound 24 (55.0 g) in acetonitrile (220 ml) and the mixture was stirred at 25-30° C. After complete deprotection of the Boc group, as monitored by HPLC, the reaction mass was filtered and the filtrate was concentrated to give H-Arg(Pbe-Pro-OAll. HCl (25, 49.64 g). HOBt (15.2 g) was added to the stirred solution of Boc-D-Arg(Pbf)-OH (26, 43.6 g) in DMF (300 ml) The reaction mixture was cooled to 0° C., and EDAC.HCl (31.76 g) and N-methylmorpholine (10.9 g) were added to it. H-Arg(Pbf)-Pro-OAll. HCl (25, 49.0 g) in DMF (165 nil) was added to the mixture stirred at 0 to 5° C. and the reaction was continued at 20 to 30° C. After completion of the reaction, as monitored by HPLC, the reaction mass was quenched with 0.5 N hydrochloric acid followed by stirring and filtration. The solid so obtained was washed with water, 7% sodium bicarbonate solution and dried to give Boc-D-Arg(Pbf)-Arg(Pbf)-Pro-OAll (27).

Yield: 55.0 g, (61.9%), Purity: 90% (HPLC)

The stirred solution of compound (27, 20.0 g) in MDC, 100 ml) was treated with tetrakis(triphenylphosphine) Palladium (0), (1.0 g) and sodium 2-ethylhexanoate (3.2 g) at 20 to 30° C. After completion of the reaction, as monitored by HPLC, the reaction mass was concentrated and residue was dissolved in DMF (60 ml) followed by addition of 1.66% Sodium bicarbonate solution and water. The resulting mass was extracted with methyl tertiary butyl ether. The organic layer was separated. Water was added to the aqueous layer followed by addition of 0.2 N hydrochloric acid till it was acidic. Stirring and filtration gave a solid which was washed with water and dried to give the tripeptide fragment C, Boc-D-Arg(Pbf)-Arg(Pbf)-Pro-OH (28).

Yield: 17.4 g, (90.38%), Purity: 88% (HPLC)

### Example 8: Preparation of Icatibant Acetate (1)

HOBt (0.74 g) was added to the stirred solution of Boc-D-Arg(Pbf)-Arg(Pbf)-Pro-OH (28, 2.53 g) in DMF (8.45 ml). The reaction mixture was cooled to 0° C., and EDAC.HCl (0.70 g) and N-methylmorpholine (0.60 g) were added to it H-Hyp-Gly-Thia-Ser(O-tBu)-D-Tic-Oic-Arg(Pbf)-OtBu (21, 3.0 g) in DMF (10.5 ml) was added to the mixture stirred at 0 to 5° C. and the reaction was continued at 20 to 30° C. After completion of the reaction, as monitored by HPLC, the reaction mass was quenched with 0.5 N hydrochloric acid followed by stirring and filtration. The solid so obtained was washed with water, 5% sodium bicarbonate solution and dried to obtain crude decapeptide, (4.26 g) which was purified on reverse phase preparative HPLC to give Boc-D-Arg(Pbf)-Arg(Pbf)-Pro-Hyp-Gly-Thia-Ser(O-tBu)-D-Tic-Oic-Arg(Pbf)-O-tBu (29).

Yield: 2.6 g, (50%), Purity: 92% (HPLC)

The solution of (29) (2.5 g) in MDC (15 ml) was stirred and trifluoroacetic acid (115 ml), triethylsilane (TES) (1.5 g) were added to it. Reaction mass was stirred at 25 to 30° C. After completion of the reaction, as monitored by HPLC, the reaction mass was concentrated and the oily residue so obtained was treated with methyl tertiary butyl ether. Stirring and filtration provided a solid which was purified on reverse phase preparative HPLC followed by treatment with acetic acid and lyophilization to give Icatibant acetate.

Yield: 0.5 g, (35%), Purity: 99.8% (HPLC).

1. A process for the solution phase synthesis of Icatibant acetate (1), comprising:
reacting H Thia-Ser(O-tBu)-D-Tic-Oic-Arg(Pbf)-O-tBu (fragment A) with Fmoc-Hyp-Gly-OH (fragment B) in presence of a coupling agent, in an organic solvent and a base to give a heptapeptide intermediate of the formula, H-Hyp-Gly-Thia-Ser(O-tBu)-D-Tic-Oic-Arg(Pbf)-O-tBu (21),
coupling H-Hyp(OP)-Gly-Thia-Ser(OP)-D-Tic-Oic-Arg(Pbf)-O-tBu (21) with Boc-D-Arg(Pbf)-Arg(Pbf)-Pro-OH (fragment C) in presence of a coupling agent, in an organic solvent and a base to provide a decapeptide of the formula Boc-D-Arg(Pbf)-Arg(Pbf)-Pro-Hyp-Gly-Thia-Ser(O-tBu)-D-Tic-Oic-Arg(Pbf)-O-tBu (29), and
subjecting Boc-D-Arg(Pbf)-Arg(Pbf)-Pro-Hyp(O-tBu)-Gly-Thia-Ser(O-tBu)-D-Tic-Oic-Arg(Pbf)-O-tBu (29) to deprotection followed by treatment with acetic acid to provide Icatibant acetate (1) having desired purity.

2. (canceled)

3. (canceled)

4. A process for the solution phase synthesis of Icatibant acetate (1) as claimed in claim 1 wherein the H-Thia-Ser(O-tBu)-D-Tic-Oic-Arg(Pbf)-OtBu (fragment A) is prepared by:
deprotecting Boc-D-Tic-OBn (2) followed by reaction with Boc-Ser(O-tBu)-OH (4) to give Boc-Ser(O-tBu)-D-Tic-OBn (5),
deprotecting Boc-Ser(O-tBu)-D-Tic-OBn (5) followed by reaction with H-Oic-OAll (7) or an acid addition salt thereof to afford Boc-Ser-(O-tBu)-D-Tic-Oic-OAll (8),
deprotecting Boc-Ser-(O-tBu)-D-Tic-Oic-OAll (8) followed by reaction with Fmoc-Thia-OH (10) to give Fmoc-Thia-Ser(O-tBu)-D-Tic-Oic-OAll (11),
deprotecting Fmoc-Thia-Ser(O-tBu)-D-Tic-Oic-OAll (11) followed by reaction with H-Arg(Pbf)-OtBu.HCl (13) to give Fmoc-Thia-Ser(O-tBu)-D-Tic-Oic-Arg(Pbf)-O-tBu (14) which on subsequent deprotection yields fragment A.

5. A process for the solution phase synthesis of Icatibant acetate (1) as claimed in claim 1 wherein the Fmoc-Hyp-Gly-OH (fragment B) is prepared by reacting H-Gly-OAll (16) or an acid addition salt thereof with Fmoc-Hyp-OH (17) to give Fmoc-Hyp-Gly-OAll (18), which on deprotection yields gave fragment B.

6. A process for the solution phase synthesis of Icatibant acetate (1) as claimed in claim 1 wherein the Boc-D-Arg(Pbf)-Arg(Pbf)-Pro-OH (fragment C) is prepared by reacting H-Pro-OAll (22) or an acid addition salt thereof with Boc-Arg(Pbf)-OH (23) to give Boc-Arg(Pbf)-Pro-OAll (24), deprotecting Boc-Arg(Pbf)-Pro-OAll (24) followed by reaction with Boc-D-Arg(Pbf)-OH (26) to give Boc-D-Arg(Pbf)-Arg(Pbf)-Pro-OAll (27), and deprotecting Boc-D-Arg(Pbf)-Arg(Pbf)-Pro-OAll (27) to yield Boc-D-Arg(Pbf)-Arg(Pbf)-Pro-OH (fragment C).

7. (canceled)

8. (canceled)

9. (canceled)

10. (canceled)

11. The process as claimed in claim 1 wherein the solvent is selected from the group consisting of methylene chloride, chloroform, dichloroethane, dimethylformamide, dimethyl sulfoxide, tetrahydrofuran, ethyl acetate, N-methyl-2-pyrrolidinone, acetonitrile and combinations thereof.

12. The process as claimed in claim 1 wherein the coupling agent is selected from the group consisting of diisopropylcarbodiimide, dicyclohexylcarbodiimide, 1-Ethyl-3-(3-dimethylaminopropyl) carbodiimide (EDAC), and BOP (Benzotriazol-1-yloxy-tris(dimethylamino)-phosphonium-hexafluoro phosphate).

13. The process as claimed in claim 1 wherein the base is selected from the group consisting of diisopropylethylamine, N-methylmorpholine, triethylamine, diethyl amine, piperidine and N-methylpyrrolidine.

14. The process as claimed in claim 4 wherein the deprotection is carried out with tetrakis(triphenylphosphine)palladium.

15. A compound selected from the group consisting of:
Boc-D-Tic-OBn (2),
Boc-Ser(O-tBu)-D-Tic-OBn (5),
Boc-Ser-(O-tBu)-D-Tic-OH (6),
Boc-Ser-(O-tBu)-D-Tic-Oic-OAll (8),
Fmoc-Thia-Ser(O-tBu)-D-Tic-Oic-OAll (11),
Fmoc-Thia-Ser(O-tBu)-D-Tic-Oic-OH (12),
Fmoc-Hyp-Gly-OAll (18),
Fmoc-Hyp-Gly-Thia-Ser(O-tBu)-D-Tic-Oic-Arg(Pbf)-O-tBu (20),
H-Hyp-Gly-Thia-Ser(O-tBu)-D-Tic-Oic-Arg(Pbf)-O-tBu (21),
Boc-Arg(Pbf)-Pro-OAll (24),
Boc-D-Arg(Pbf)-Arg(Pbf)-Pro-OAll (27),
H-Thia-Ser(O-tBu)-D-Tic-Oic-Arg(Pbf)-O-tBu (Fragment A),
Fmoc-Hyp-Gly-OH (Fragment B), and
Boc-D-Arg(Pbf)-Arg(Pbf)-Pro-OH (Fragment C).

16. (canceled)

17. The process as claimed in claim 5 wherein the deprotection is carried out with tetrakis(triphenylphosphine)palladium.

18. The process as claimed in claim 6 wherein the deprotection is carried out with tetrakis(triphenylphosphine)palladium.

19. A process for the synthesis of Icatibant acetate (1) comprising:
(i) deprotecting Boc-D-Tic-OBn (2);
(ii) reacting the deprotected Boc-D-Tic-OBn (2) with Boc-Ser(O-tBu)-OH (4) to yield Boc-Ser(O-tBu)-D-Tic-OBn (5);
(iii) deprotecting Boc-Ser(O-tBu)-D-Tic-OBn (5);
(iv) reacting the deprotected Boc-Ser(O-tBu)-D-Tic-OBn (5) with H-Oic-OAll (7) or an acid addition salt thereof to afford Boc-Ser-(O-tBu)-D-Tic-Oic-OAll (8);
(v) deprotecting Boc-Ser-(O-tBu)-D-Tic-Oic-OAll (8);
(vi) reacting the deprotected Boc-Ser-(O-tBu)-D-Tic-Oic-OAll (8) with Fmoc-Thia-OH (10) to yield Fmoc-Thia-Ser(O-tBu)-D-Tic-Oic-OAll (11);
(vii) deprotecting Fmoc-Thia-Ser(O-tBu)-D-Tic-Oic-OAll (11);
(viii) reacting the deprotected Fmoc-Thia-Ser(O-tBu)-D-Tic-Oic-OAll (11) with H-Arg(Pbf)-OtBu.HCl (13) to yield Fmoc-Thia-Ser(O-tBu)-D-Tic-Oic-Arg(Pbf)-O-tBu (14);
(ix) deprotecting Fmoc-Thia-Ser(O-tBu)-D-Tic-Oic-Arg(Pbf)-O-tBu (14) to yield H-Thia-Ser(O-tBu)-D-Tic-Oic-Arg(Pbf)-O-tBu (fragment A);
(x) reacting H-Thia-Ser(O-tBu)-D-Tic-Oic-Arg(Pbf)-O-tBu (fragment A) with Fmoc-Hyp-Gly-OH (fragment B) in presence of a coupling agent, in an organic solvent and a base to yield H-Hyp-Gly-Thia-Ser(O-tBu)-D-Tic-Oic-Arg(Pbf)-O-tBu (21);
(xi) coupling H-Hyp(OP)-Gly-Thia-Ser(OP)-D-Tic-Oic-Arg(Pbf)-O-tBu (21) with Boc-D-Arg(Pbf)-Arg(Pbf)-Pro-OH (fragment C) in presence of a coupling agent, in an organic solvent and a base to provide a decapeptide of the formula Boc-D-Arg(Pbf)-Arg(Pbf)-Pro-Hyp-Gly-Thia-Ser(O-tBu)-D-Tic-Oic-Arg(Pbf)-O-tBu (29).

20. The process for the synthesis of Icatibant acetate as claimed in claim 19 wherein the Fmoc-Hyp-Gly-OH (fragment B) is prepared by reacting H-Gly-OAll (16) or an acid addition salt thereof with Fmoc-Hyp-OH (17) to give Fmoc-Hyp-Gly-OAll (18), which on deprotection yields fragment B.

21. The process for the synthesis of Icatibant acetate as claimed in claim 19 wherein the Boc-D-Arg(Pbf)-Arg(Pbf)-Pro-OH (fragment C) is prepared by reacting H-Pro-OAll (22) or an acid addition salt thereof with Boc-Arg(Pbf)-OH (23) to yield Boc-Arg(Pbf)-Pro-OAll (24), deprotecting Boc-Arg(Pbf)-Pro-OAll (24) followed by reaction with Boc-D-Arg(Pbf)-OH (26) to give Boc-D-Arg(Pbf)-Arg(Pbf)-Pro-OAll (27), and deprotecting Boc-D-Arg(Pbf)-Arg(Pbf)-Pro-OAll (27) to yield Boc-D-Arg(Pbf)-Arg(Pbf)-Pro-OH (fragment C).

22. The process for the synthesis of Icatibant acetate as claimed in claim 19 wherein:
the solvent is selected from the group consisting of methylene chloride, chloroform, dichloroethane, dimethylformamide, dimethyl sulfoxide, tetrahydrofuran, ethyl acetate, N-methyl-2-pyrrolidinone, acetonitrile and combinations thereof;
the coupling agent is selected from the group consisting of diisopropylcarbodiimide, dicyclohexylcarbodiimide, 1-Ethyl-3-(3-dimethylaminopropyl) carbodiimide (EDAC), and BOP (Benzotriazol-1-yloxy-tris(dimethylamino)-phosphonium-hexafluoro phosphate);
the base is selected from the group consisting of diisopropylethylamine, N-methylmorpholine, triethylamine, diethyl amine, piperidine and N-methylpyrrolidine; and
deprotection is carried out with tetrakis(triphenylphosphine)palladium.
